(19) United States
(12) Patent Application Publication
(10) Pub. No.: US 2010/0047173 A1
(43) Pub. Date: Feb. 25, 2010

(54) Methods for Using Optical Agents

(76) Inventors: Richard B. Dorshow, St. Louis, MO (US); Raghavan Rajagopalan, Solon, OH (US); Dennis A. Moore, St. Louis, MO (US)

Correspondence Address: Mallinckrodt Inc., 675 McDonnell Boulevard, HAZELWOOD, MO, 63042, US

(21) Appl. No.: 12/515,294

(22) PCT Filed: Nov. 13, 2007
(86) PCT No.: PCT/US07/24066

§ 371 Date: May 18, 2009

Related U.S. Application Data
(60) Provisional application No. 60/860,270, filed on Nov. 21, 2006.

(51) Int. Cl. A61K 49/00

(52) U.S. Cl. 424/9.1

(57) ABSTRACT

In certain embodiments, the present invention is directed to novel processes for using hepatobiliary cleared optical agents to detect one or more tissues of the biliary tract of a surgical patient. In certain embodiments, the invention is directed to kits that include one or more optical agents and instructions for using the agent(s), for example, in a process of the invention.

### FIELD OF THE INVENTION

The present invention generally relates to the use of optical agents in medical imaging of patients.

### BACKGROUND

Surgical procedures can require intricate manipulations within a confined area where it may be difficult to differentiate between structures for surgical intervention from structures that should remain undisturbed. As a result, abdominal surgeries carry a risk of accidental injury to several organs, including liver, gallbladder and numerous ducts connecting the two organs such as bile, hepatic and cystic ducts. The bile produced in the liver is collected in bile canaliculi, which merge to form bile ducts. These eventually drain into the right and left hepatic ducts, which in turn merge to form the common hepatic duct. The cystic duct from the gallbladder joins with the common hepatic duct to form the common bile duct (CBD). Bile can either drain directly into the duodenum via the common bile duct or be temporarily stored in the gallbladder via the cystic duct. The common bile duct and the pancreatic duct enter the duodenum together at the ampulla of Vater.

Approximately ten percent of Americans will develop gallstone disease (cholelithiasis) in their lifetime. Most often the stones cause no symptoms and their presence goes unrecognized. The most common symptom complex is biliary colic, characterized by abdominal pain localized to the right upper abdomen, which often follows large or excessively fatty meals. Patients usually improve without intervention, but bouts often recur. Gallstones may also cause other concerns including cholecystitis (infection of the gallbladder), gallstone pancreatitis (inflammation of the pancreas), jaundice, or cholangitis (infection of the ducts connecting the gallbladder with the liver and small intestine). Medical evidence exists to suggest that long-standing gallstone disease may eventually lead to cancer of the gallbladder, a very aggressive and often deadly tumor. Thus, in a number of patients gallstones present an indication for gallbladder removal, i.e., cholecystectomy. Other indications for cholecystectomy include prophylactic removal of the gallbladder in patients with cholelithiasis who are scheduled to undergo organ transplantation, or in patients with a calcified (porcelain) gallbladder, thought to be associated with gallbladder cancer. Rarer indications include trauma, biliary dyskinesis, and symptomatic gallbladder polyps.

Due to faster recuperation and shorter hospital stay, laparoscopic cholecystectomy (LC) has replaced the open cholecystectomy as a standard procedure. However, while beneficial in many ways, LC has led to an increase in bile duct injuries. See, e.g., Strasberg S M, Hertl M, Soper N J. An analysis of the problem of biliary injury during laparoscopic cholecystectomy. J Am Coll Surg 1995; 180:101-25. This seems partly related to the different anatomical exposure of the area around the gallbladder, especially the Calot's triangle, during the laparoscopic procedure as opposed to the open procedure. The upper border of Calot's triangle is formed by the inferior surface of the liver with the other two boundaries being the cystic duct and the bile duct. Its contents usually include the RHA, the cystic artery, the cystic lymph node (of Lund), connective tissue, and lymphatics. Occasionally it may contain accessory hepatic ducts and arteries. During cholecystectomy, the Calot's triangle is dissected to identify the cystic artery and cystic duct before ligation and division. In reality, it may be a small space rather than a large triangle, thus making the dissection of its contents without damaging the bordering structures a challenging step of a cholecystectomy.

Prevention of injury to the ductal system continues to be a matter of considerable concern of surgeons performing cholecystectomy, especially where cholecystectomy is performed laparoscopically. A few methods have been practiced to prevent injuries such as using a 30° laparoscope, applying a three dimensional laparoscope, and inserting a laparoscope through the right side of umbilicus. Greater efforts have been concentrated on dealing with the uncertain anatomy. Currently, among the primary means of preventing injury resulting from uncertain anatomy are careful dissection, the judgment of an experienced surgeon, conversion to open cholecystectomy and intraoperative cholangiography (IOC).

IOC depends on the radiopaque dye introduced into the ductal system via the cystic duct and displayed by either a static film or fluoroscopy which does not always identify the relationship of the ductal system to adjacent anatomy. The primary purpose of IOC is to identify anatomy and any aberration as well as to identify stones. The image of IOC obtained from static film or fluoroscopy is completely different from that obtained from the monitor and can not really tell where the cystic duct or common bile duct is. The information afforded by IOC can only help operators realize if there are continuity, stones, tumor and injury of the ducts but can not help them dissect easily and safely. Thus, it is of limited value during dissection of the area.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

One aspect of the present invention is directed to a process for using an optical agent in a surgical procedure. In this process, a hepatobiliary cleared optical agent (i.e., an optical agent that is at least partially cleared from the body via the liver) is administered to a patient to cause the optical agent to appear in the patient's biliary tract (e.g., liver, cystic duct, hepatic duct, common bile duct, gall bladder). A first tissue of the patient's biliary tract is irradiated with non-ionizing radiation (i.e., electromagnetic radiation that does not carry enough energy to completely remove at least one electron from an atom or molecule of the patient's body) to enable detection of the optical agent therein, and thus, demarcate the position of the first tissue.

Another aspect of the invention is directed to a process for using an optical agent in a surgical procedure. In this process, a surgical field of a patient is irradiated with non-ionizing radiation to detect a hepatobiliary cleared optical agent located in a first tissue of the biliary tract in the surgical field of the patient. A second tissue of the patient is then surgically manipulated based, at least in part, on the optical detection of the agent in the first tissue.

Still another aspect of the invention is directed to a process for using an optical agent in a surgical procedure. In this process, a hepatobiliary cleared optical agent is delivered (e.g., by way of intravenous injection) to a first tissue of a biliary tract of a patient. At least the first tissue is irradiated with non-ionizing radiation to detect the optical agent in the patient. Based at least in part on the optical agent detected, a determination may be made as to whether or not the agent is retained within the first tissue of the biliary tract.

The present invention is further directed to kits. An exemplary kit of the invention includes a hepatobiliary cleared optical agent (e.g., as a component of a biocompatible composition), and instructions for using the agent to optically detect a tissue of the biliary tract of a patient. For instance, the instructions may include instructions to carry out any of the processes of the invention described herein.

It should be noted that any of a number of appropriate agents may be utilized in kits and processes of the present invention. For instance, examples of appropriate optical agents may include, but are not limited to, cyanines, indocyanines, phthalocyanines, porphyrins, rhodamines, phenoxazines, phenothiazines, phenoselenazines, fluoresceins, benzoporphyrins, squaraines, corrins, croconiums, borondipyrroles, acridines, acridones, anthraquinones, anthracyclines, pyrazines, azaphenanthrenes, chalcogenopyrylium analogues, chlorins, naphthalocyanines, triayrlmethines, indolenium compounds, azo compounds, and diazo compounds.

Various refinements exist of the features noted above in relation to the various aspects of the present invention. Further features may also be incorporated in these various aspects as well. These refinements and additional features may exist individually or in any combination. For instance, various features discussed below in relation to one or more of the exemplary embodiments may be incorporated into any of the above-described aspects of the present invention alone or in any combination. Again, the brief summary presented above is intended only to familiarize the reader with certain aspects and contexts of the present invention without limitation to the claimed subject matter.

### BRIEF DESCRIPTION OF THE FIGURES

FIG. 1 depicts green fluorescence from fluorescein traveling through a cystic duct in an anesthetized pig. The image was taken during open surgery, post-IV administration of 6 mL of a 20 mg/mL concentration of fluorescein in PBS.

FIG. 2 depicts an ex-vivo intestine of a rat showing feces that fluoresce due to the ICG within. Blue is indicative of high fluorescence, and red is indicative of low fluorescence.

FIG. 3 depicts in vivo fluorescence time dependence after a bolus injection with ICG for rats having partial hepatectomies (impaired) and for a rat having normal liver function. The solid lines are single exponential fits to the measured data.

### DETAILED DESCRIPTION OF EXEMPLARY EMBODIMENTS

In accordance with the present invention, one or more optical agents that are at least partially hepatobiliary cleared are administered to a surgical patient to cause the optical agent(s) to appear in the patient's biliary tract. Once there, the optical agent(s) may be detected by irradiating one or more tissues of the biliary tract with non-ionizing radiation thereby demarcating tissues for a variety of medical uses.

One aspect of the present invention is directed to the appearance of an optical agent in the biliary tract of a patient, which permits a surgeon or other health care professional to readily distinguish tissue of the biliary tract from surrounding tissues. The surgeon can thus avoid accidental injury to the biliary tract (e.g., nicking or severing of the cystic duct) during a surgical procedure involving a nearby organ or tissue.

In another aspect, the appearance of an optical agent in the biliary tract of a patient may permit a surgeon or other health care professional to identify and/or delimit the target of the surgical procedure. For example, during a cholecystectomy or other surgical procedure involving one or more tissues of the biliary tract, the surgeon or other health care professional may readily identify the common bile duct or other hepatobiliary tissue by detection of the optical agent contained therein.

In yet another aspect, the appearance of an optical agent permits a surgeon or other health care professional to assess the integrity of one or more tissues of the biliary tract. For example, if an optical agent remains confined within the biliary tract upon completion of a surgical procedure, this indicates that the ducts of the biliary tract were not nicked during the surgical procedure and that the integrity of the common bile duct and other tissues of the biliary tract has not been compromised. In contrast, if the cystic duct or other tissue of the biliary tract has been damaged, the surgeon or other health care professional may readily identify the location of such damage (e.g., by observing the egress of dye from the site of the damage).

In general, the patient is a human or other warm-blooded animal that is a candidate for, is undergoing, or has undergone any surgical procedure involving a tissue or organ located in the abdominal region and/or wherein the abdominal cavity is at least partially penetrated. In some embodiments, the patient is a human patient. In some embodiments, the patient is a non-human animal undergoing abdominal surgery. For example, in a human patient undergoing a cholecystectomy, a process of the present invention may be used to avoid accidental injury to the common bile duct, cystic duct, and/or other tissues of the biliary tract.

### I. Surgical Procedures

In general, the optical agent may be used in conjunction with a range of surgical methods. For example, the optical agent may be used in “open” procedures or in minimally invasive surgeries, sometimes referred to as bandaid or keyhole surgeries. In open procedures, an incision sufficiently large to expose the entire operative area is made with a scalpel or other knife. In minimally invasive surgeries, one or more much smaller incisions are typically made, through which a laparoscope and/or other endoscopic tools may be inserted to allow a surgeon to view and/or surgically manipulate a patient's organs or tissues.

Surgical procedures in which processes of the present invention can be used to aid a surgeon include, but are not limited to; for example, cholecystectomy, liver transplantation, liver resection, total or partial hysterectomy, hernia repair surgery, colectomy, appendectomy, splenectomy, distal or total pancreatectomy, the Whipple procedure, removal of inflammatory or malignant tumors in the abdominal regions, abdominal lymphadenectomy (removal of lymph nodes), and other surgical procedures performed in the abdominal region. In particular, processes of the present invention are particularly useful in hepatobiliary surgery including cholecystectomy, liver transplantation, liver resection, distal or total pancreatectomy, the Whipple procedure or any other procedure involving liver, gallbladder, bile ducts or pancreas.

To various degrees, these and other surgical procedures performed in the abdomen carry a risk of accidental damage to tissue of the biliary tract. The risk of damage may be especially high in laparoscopic surgical procedures, because the surgeon tends to have a limited view of the surgical area and is generally unable to use tactile perception to identify these structures. In some embodiments of the invention, one or more optical agents are administered to the patient to avoid such accidental damage by permitting a surgeon to distinguish tissue of the biliary tract from adjacent (e.g., surrounding) tissue. For example, a process of the invention may permit a surgeon to distinguish tissue of the biliary tract from tissue of the digestive tract or the spleen. As another example, a process of the invention may permit a surgeon to distinguish one or more tissues of the biliary tract from nearby arteries, veins, lymphatic vessels, and/or other tissue.

As previously noted, one aspect of the present invention relates to the use of one or more optical agents to demarcate at least one tissue of the biliary tract of a patient during a surgical procedure. For example, a process of the present invention can be used to enable the surgeon or other healthcare individual to avoid the common bile duct or any of the biliary tree ducts. Uptake of the optical agent into hepatocytes can be mediated by passive or carrier processes. Once in the parenchymal cell of the liver, the optical agent can be metabolized or bind to intracellular proteins, following which it may return to the circulation or exit from the hepatocyte into the bile canaliculus, again by passive or carrier-mediated transport, before secretion in bile.

Another aspect of the invention relates to the use of one or more optical agents to demarcate the target of a surgical procedure. Such surgical procedures include, but are not limited to, for example, liver resection, liver transplantation, pancreas resection or cholecystectomy. Still another aspect of the invention is the use of the optical agent(s) to assess the integrity of the biliary tract. Such an assessment can be made before, during, and/or after a surgical procedure performed on the biliary tract or other organ or tissue in the abdominal region. Confinement of the optical agent to the biliary tract indicates that no damage to the biliary tract (e.g., nicking of the common bile duct) has occurred. If damage or injury to the biliary tract has occurred, the process of the present invention allows a surgeon to rapidly identify the location of such damage or injury (e.g., by observing egress of the optical agent from the site of damage).

Yet another aspect of the invention relates to the use of an optical agent to detect one or more tissues of the biliary tract during a diagnostic procedure. Depending upon the surgical technique employed, the presence of the optical agent in a first tissue may be detected by irradiating the entire surgical field. This approach could be used, for example, in open surgical procedures. Alternatively, only a portion of the surgical field or the specific site(s) to be monitored may be illuminated, for example, using a laparoscope or other endoscopic tool.

In general, any source of irradiation capable of providing non-ionizing radiation of a desired wavelength (which may refer to a single wavelength, multiple wavelengths, or even one or more ranges of appropriate wavelengths) may be used. For example, in some embodiments, the operating room lighting (e.g., fluorescent or incandescent lighting) may emit light of a desired wavelength. In some embodiments, the source of irradiation may be a laser. In yet another embodiment, the source of irradiation may be a hand-held light. Other sources of irradiation that can be used include, but are not limited to, lighted catheters, endoscopes, fiber optic probes, light emitting diodes (LEDs), lighted headbands (also called headlights), and the like. A surgical instrument that contains or is equipped with an illumination system may also be employed. Examples of such instruments include fiber optic instruments available from BioSpec (Moscow, Russia) and the TC-I fiber optic tool for photodynamic therapy having a fine needle tip for irradiating interstitial tumors (http://www.biospec.ru/_Fiber_Optics_e.html).

Any optical detection methods available in the art can be used in processes of the present invention to detect the optical agent in the biliary system. Spectroscopic measurements tend to be separated into three broad categories: absorbance, scattering/reflectance, and emission. Absorbance assays involve relating the amount of incident light that is absorbed by a sample to the type and number of molecules in the sample. For example, in the case of an absorbance measurement, it is desirable that the non-ionizing radiation that is used include at least one wavelength that is absorbed by the optical agent. Most commonly, absorbance is measured indirectly by studying the portion of incident light that emerges from the sample. Scattering assays are similar to absorbance in that the measurement is based on the amount of incident light that emerges or is transmitted from the sample or tissue. However, in the case of scattering, the signal increases with the number of interactions, whereas, in the case of absorbance, the signal is inversely proportional to the interactions. Emission assays look at electromagnetic emissions from a sample other than the incident light. In each case, the measurements may be a broad spectrum or frequency-specific depending on the particular assay. Most commonly, emission assays involve the measurement of luminescence.

Luminescence is the emission of light from excited electronic states of atoms or molecules. Luminescence generally refers to all kinds of light emission, except incandescence, and may include photoluminescence, chemiluminescence, and electrochemiluminescence, among others. In photoluminescence, including fluorescence and phosphorescence, the excited electronic state is created by absorption of electromagnetic radiation. Luminescence assays involve detection and interpretation of one or more properties of the luminescence or associated luminescence process. These properties include intensity, excitation and/or emission spectrum, polarization, lifetime, and energy transfer, among others. These properties also include time-independent (steady-state) and/or time-dependent (time-resolved) properties of the luminescence. Representative luminescence assays include fluorescence intensity (FLINT), fluorescence polarization (FP), fluorescence resonance energy transfer (FRET), fluorescence lifetime (FLT), total internal reflection fluorescence (TIRF), fluorescence correlation spectroscopy (FCS), fluorescence recovery after photobleaching (FRAP), and bioluminescence resonance energy transfer (BRET), among others. By way of example, when a fluorescent optical agent is used in the present invention, it is desirable that the wavelength of non-ionizing radiation be such that it excites the optical agent. This excitation causes the molecule to emit part of the absorbed energy at a different wavelength, and the emission can be detected using fluorometric techniques as described above. One skilled in the art can readily determine the most appropriate detection technique based on, in part, the specific optical agent(s) administered, the tissue to be detected, and the type of surgical procedure involved. For example, in some embodiments, the surgeon will be able to see the optical agent in the surgical field. Other embodiments employ an optical agent that can be detected using a laparoscopic instrument.

Upon irradiation with electromagnetic radiation of the proper wavelength, an optical agent may be detected by visual or other optical means. For example, optical detection may be achieved using the unaided eye or by one or more imaging or detecting devices (e.g., a camera, charged coupled device (CCD), photomultiplier tube (PMT), avalanche diode, photodiodes, endoscope, laparoscope), or detection involving an electronic processing step (e.g., detecting, enhancing, processing, analyzing, quantitating, or otherwise manipulating a signal using software or other means). For example, an electronic detecting device may be utilized to detect luminescence being emitted from the optical agent within the biliary tract tissue. The luminescence detected may be converted or otherwise processed into an electronic signal that may be displayed as an image (e.g., on a computer screen or other appropriate display) and/or displayed as one or more data points.

### II. Dyes/Optical Agents

Optical agents (also referred to as optical dyes) used in processes of the invention are at least partially hepatobiliary cleared. That is, upon administration to a patient, at least a fraction of the administered dose of the optical agent will be excreted by way of the biliary tract (i.e., via secretion into bile). In general, the size and hydrophobicity of a pharmaceutical or diagnostic agent influences the route by which it is excreted when it is administered to a patient. Small, hydrophilic molecules tend to be excreted via the renal system, whereas larger, hydrophobic molecules tend to be excreted via the hepatobiliary route. Thus, in general, optical agents employed in processes of the invention may tend to be relatively large in size and/or relatively more hydrophobic than dyes excreted predominantly via the renal route. The optical agents may be coupled or associated with moieties which render them more hydrophobic and thus increase their capacity to be excreted via the biliary tract. For example, a strong binding to human serum albumin has been observed by using to phenyl rings attached to a cydohexyl moiety, in particular, diphenylcydohexyl (see, e.g., The Chemistry of Contrast Agents in Medical Magnetic Resonance Imaging, Edited by Andre E. Merback and Eva Toth, John Wiley & Sons, Chichester, 2001, Chapter 5). The degree to which an optical agent is hepatobiliarily excreted can be determined empirically by those skilled in the art. Examples of such systems include isolated perfused rat liver (IPRL), and bile duct cannulated (BDC) rat models (see, e.g., Chan et al., DDT (1996) 1:461-473). In healthy human subjects, feces typically are used as a surrogate to quantify the amount of drug excreted via nonurinary pathways.

Optical agents used in processes of the invention include those that are already at least partially excretable by a hepatobiliary route as well as those that are rendered at least partially excretable by a hepatobiliary route (e.g., by including one or more lipophilic substituents, and/or by removing or blocking hydrophilic substituents). These optical agents are preferably untargeted. That is, these optical agents are preferably not associated with a carrier or conjugate which increases the selectivity of the optical agent for localization in a particular organ or tissue.

Hepatobiliary cleared optical agents used in processes the invention tend to be chromophores, fluorophores, and/or the like. Optimal absorption or excitation maxima for optical agents will vary depending on the optical agent employed, but in general, preferred optical agents tend to absorb or be excited by light in the ultraviolet (UV), visible, or infrared (IR) range of the electromagnetic spectrum. For example, the non-ionizing radiation employed in the process of the present invention may range in wavelength from about 350 nm to about 1200 nm. In some embodiments, in may be desirable to simply utilized visible and/or near infrared light.

ICG is known to be cleared from the blood stream by the liver and excreted into the bile. Other exemplary lipophilic optical agents include, but are not limited to, acridine orange (3,6-bis[dimethylamino]acridinium chloride hemi[zinc chloride salt]), lipophilic azo dyes, unhalogenated naphthalimide dyes, non-azo 1,8-naphthalimide dyes, diphenylhexatriene, phenoxazine dyes (e.g., Nile Red), N-phenyl-1-naphthylamine, Prodan, Laurodan, Pyrene, Perylene, rhodamine, rhodamine B, tetramethylrhodamine, Texas Red, octadecyl rhodamine B, sulforhodamine, lipophilic carbanocyanines (e.g., DiO (3,3′-dioctadecyloxacarbocyanine perchlorate), DiI (1,1′-dioctadecyl-3,3,3′,3′-tetramethylindocarbocyanine perchlorate) (orange fluorescence), DiO (a dialkyl carbocyanine dye exhibiting green fluorescence), DiD (1,1′-dioctadecyl-3,3,3′,3′′-tetramethylindodicarbocyanine perchlorate) (red fluorescence), DiR (a dialkyl carbocyanine dye exhibiting infrared fluorescence) Dilinoleyl DiI (also called FAST DiI1 or 1,1′-dilinoleyl-3,3,3′,3′-tetramethylindocarbocynanine perchlorate), Dilinoleyl DiO (also called FAST DiO1, or 1,1′-dilinoleyl-3,3′-oxacarbocynanine perchlorate), DiOC₁₄(3), hydroxyethanesulfonate (3,3′-ditetra decyloxacarbocyanine, hydroxyethanesulfonate), DiR (DiIC₁₈(7) or 1,1′-dioctadecyl-3,3,3′,3′-tetramethyl indotricarbocyanine iodide),and combinations thereof (Molecular Probes, Eugene Oreg.), BODIPY FL (a borondipyrrole dye exhibiting green fluorescence), BODIPY 558/568 BFA (a borondipyrrole dye exhibiting red orange fluorescence), cholephilic dipyrrinones such as highly fluorescent N,N′-carbonyl-bridged analogue of xanthobilirubic acid (xanthoglow) as disclosed in Woydziak et al., Synthesis and Hepatic Transport of Strongly Fluorescent Cholephilic Dipyrrinones, J Org Chem 70;8417 (2005), and others as known to one skilled in the art. The appropriate activation energy for each dye is known, or is readily determined by one skilled in the art, based on the absorption/emission spectra of the dye. In preparations labeled with dyes having multiple emission profiles, the following dyes can be used: DiI exciting at 568 nm (red), DiO at 488 nm (green) and DiD at 647 nm (blue). Combinations of carbocyanine dyes may be used, e.g., 1 DiI:1 DiO, 1 DiI:1 DiD and 1 DiO:1 DiD.

With respect to a number of optical agents, it is known that absorption and emission properties can be affected, for example, by one or more of the concentration of the agent, the solvent in which the agent is dissolved and/or suspended, the wavelength of the excitation light, and the distance between the source and a detector. However, a skilled artisan can readily determine the optimal absorption and emission properties for an optical agent used herein. For example, ICG has an absorption peak at the wavelength of about 780 nm and an emission peak at the wavelength of about 830 nm in a dilute aqueous solution. With increasing ICG concentration, the absorption peak tends to shift to a wavelength of about 695 nm.

One exemplary family of optical agents that may be utilized in processes of the invention include optical agents corresponding to formula 1 below, wherein

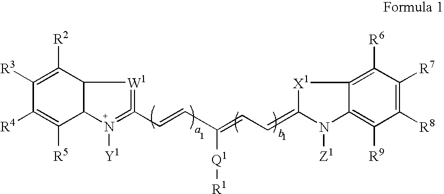

W¹ and X¹ may be the same or different and are selected from the group consisting of —CR^{w}R^{x}, —O—, —NR^{y}, —S—, and —Se—; Q² is a single bond or is selected from the group consisting of —O—, —S—, —Se—, and —NR⁵; a₁ and b₁ independently vary from 0 to 5; a and c are independently from 1 to 20; b and d are independently from 1 to 100; Y¹ is a constituent selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R^{y})—(CH₂)_{b}—CONH₂, (CH₂)ₐ—N(R^{y})—(CH₂)_{c}—NHCOH, —(CH₂)ₐ—N(R^{y})—CH₂—(CH₂OCH₂)_{b}—CH₂—CONH₂, —(CH₂)ₐ—N(R^{y})—CH₂—(CH₂OCH₂)_{b}—CH₂—NHCOH, —CH₂—(CH₂OCH₂)_{b}—CH₂—N(R^{y})—(CH₂)ₐ—CONH₂, —CH₂—(CH₂OCH₂)_{b}—CH₂N(R^{y})—(CH₂)ₐ—NHCOH, —CH₂—(CH₂OCH₂)_{b}—CH₂—N(R^{y})—CH₂—(CH₂OCH₂)_{d}—CONH₂, —CH₂(CH₂OCH₂)_{b}—CH₂NR^{y}R^{z} —CH₂—(CH₂OCH₂)_{b}—CH₂—N(R^{y})—CH₂—(CH₂OCH₂)_{d}—NHCOH, AND —(CH₂)ₐ—NR^{y}R^{z};
Z¹ is a constituent selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂(CH₂OCH₂)_{b}—CH₂—NHCOH, —(CH₂)ₐ—N(R^{y})—(CH₂)_{b}—CONH₂, (CH₂)ₐ—N(R^{y})—(CH₂)_{c}—NHCOH, —(CH₂)ₐ—N(R^{y})—CH₂—(CH₂OCH₂)_{b}—CH₂—CONH₂, —(CH₂)ₐ—N(R^{y})—CH₂—(CH₂OCH₂)_{b}—CH₂—NHCOH, —CH₂—(CH₂OCH₂)_{b}—CH₂—N(R^{y})—(CH₂)ₐ—CONH₂, —CH₂—(CH₂OCH₂)_{b}—CH₂—N(R^{y})—(CH₂)ₐ—NHCOH, —CH₂—(CH₂OCH₂)_{b}—CH₂—N(R^{y})—CH₂—(CH₂OCH₂)_{d}—CONH₂, —CH₂—(CH₂OCH₂)_{b}—CH₂—N(R^{y})—CH₂—(CH₂OCH₂)_{d}—NHCOH, —(CH₂)ₐ—NR^{y}R^{z}, and —CH₂(CH₂OCH₂)_{b}—CH₂NR^{y}R^{z}; R^{w}, R^{x}, R^{y}, R^{z}, and R¹ to R⁹ are independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, glucose derivatives of R groups, cyano, nitro, halogen, saccharide, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—OH and —CH₂—(CH₂OCH₂)_{b}—CO₂H.

Another exemplary family of optical agents that may be utilized in processes of the invention include optical agents corresponding to formula 2 below, wherein

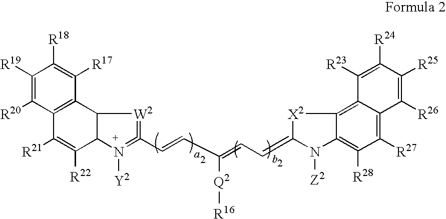

W² and X² may be the same or different and are selected from the group consisting of —CR¹R², —O—, —NR³, —S—, and —Se—; Q² is a single bond or is selected from the group consisting of —O—, —S—, —Se—, and —NR⁵; a₂ and b₂ independently vary from 0 to 5; a and c are independently from 1 to 20; b and d are independently from 1 to 100; Y² is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂—(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; Z² is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂—(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; R¹ to R⁵, and R¹⁶ to R²⁸ are constituents independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, glucose derivatives of R groups, cyano, nitro, halogen, saccharide, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂NHCOH, —(CH₂)ₐ—OH and —CH₂—(CH₂OCH₂)_{b}—CO₂H.

Still another exemplary family of optical agents that may be utilized in processes of the invention include optical agents corresponding to formula 3 below, wherein

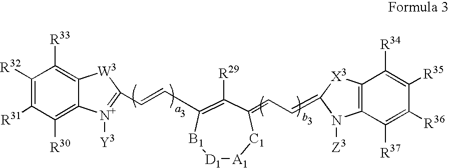

W³ and X³ may be the same or different and are selected from the group consisting of —CR¹R², —O—, —NR³, —S—, and —Se; Y³ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R³)—(CH₂)_{b}—CONH₂, —(CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂—(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; Z³ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂(CH₂OCH₂)_{b}—CH₂—NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂(CH₂OCH₂)_{b}—CH₂—N(R³)—(CH₂)ₐ—CONH₂, —CH₂—(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; A₁ is a single or a double bond; B₁, C₁, and D₁ may the same or different and are selected from the group consisting of —O—, —S—, —Se—, —P—, —CR¹R², —CR¹, alkyl, NR³, and —C═O; A₁, B₁, C₁, and D₁ may together form a 6- to 12-membered carbocyclic ring or a 6- to 12-membered heterocyclic ring optionally containing one or more oxygen, nitrogen, or sulfur atom; a₃ and b₃ independently vary from 0 to 5; R¹ to R⁴, and R²⁹ to R³⁷ are independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, glucose derivatives of R groups, cyano, nitro, halogen, saccharide, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—OH and —CH₂—(CH₂OCH₂)_{b}—CO₂H; a and c are independently from 1 to 20; and b and d are independently from 1 to 100.

Yet another exemplary family of optical agents that may be utilized in processes of the invention include optical agents corresponding to formula 4 below, wherein

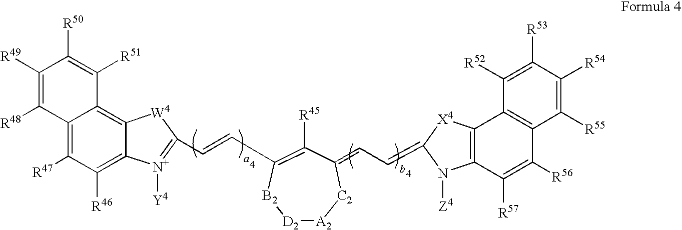

W⁴ and X⁴ may be the same or different and are selected from the group consisting of —CR¹R², —O—, —NR³, —S—, and —Se; Y⁴ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂—(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; Z⁴ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂—(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂—(CH₂OCH₂)_{b}—CH₂N(R³)—CH₂—(CH₂OCH₂)_{d}—NHCOH, —(CH₂)ₐ—NR³R⁴, and —CH₂(CH₂OCH₂)_{b}—CH₂NR³R⁴; A₂ is a single or a double bond; B₂, C₂, and D₂ may be the same or different and are selected from the group consisting of —O—, —S—, —Se—, —P—, —CR¹R², —CR¹, alkyl, NR³, and —C═O; A₂, B₂, C₂, and D₂ may together form a 6- to 12-membered carbocyclic ring or a 6- to 12-membered heterocyclic ring optionally containing one or more oxygen, nitrogen, or sulfur atom; a₄ and b₄ independently vary from 0 to 5; R¹ to R⁴, and R⁴⁵ to R⁵⁷ are independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, glucose derivatives of R groups, cyano, nitro, halogen, saccharide, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂CONH₂, —(CH₂)ₐ—NHCOH, —CH₂(CH₂OCH₂)_{b}—CH₂—NHCOH, —(CH₂)ₐ—OH and —CH₂—(CH₂OCH₂)_{b}—CO₂H; a and c are independently from 1 to 20; and b and d are independently from 1 to 100.

Still yet another exemplary family of optical agents that may be utilized in processes of the invention include optical agents corresponding to formula 5 below, wherein

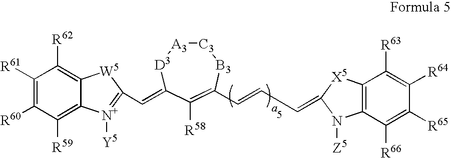

W⁵ and X⁵ may be the same or different and are selected from the group consisting of —CR¹R², —O—, —NR³, —S—, and —Se; Y⁵ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂(CH₂OCH₂)_{b}—CH₂CONH₂, —(CH₂)ₐ—NHCOH, —CH₂—(CH₂OCH₂)_{b}—CH₂NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂NHCOH, —CH₂—(CH₂OCH₂)_{b}—CH₂—N(R³)—(CH₂)ₐ—CONH₂, —CH₂(CH₂OCH₂)_{b}—CH₂N(R³)—(CH₂)ₐ—NHCOH, —CH₂(CH₂OCH₂)_{b}—CH₂N(R³)—CH₂—(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; Z⁵ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂(CH₂OCH₂)_{b}—CH₂NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂—(CH₂OCH₂)_{b}—CH₂N(R³)—(CH₂)ₐ—NHCOH, —CH₂—(CH₂OCH₂)_{b}—CH₂N(R³)—CH₂(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; A₃ is a single or a double bond; B₃, C₃, and D₃ may be the same or different and are selected from the group consisting of —O—, —S—, —Se—, —P—, —CR¹R², —CR¹, alkyl, NR³, and —C═O; A₃, B₃, C₃, and D₃ may together form a 6- to 12-membered carbocyclic ring or a 6- to 12-membered heterocyclic ring optionally containing one or more oxygen, nitrogen, or sulfur atom; a₅ is independently from 0 to 5; R¹ to R⁴, and R⁵⁸ to R⁶⁶ are independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, glucose derivatives of R groups, cyano, nitro, halogen, saccharide, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—OH and —CH₂—(CH₂OCH₂)_{b}—CO₂H; a and c are independently from 1 to 20; and b and d are independently from 1 to 100.

Even still another exemplary family of optical agents that may be utilized in processes of the invention include optical agents corresponding to formula 6 below, wherein

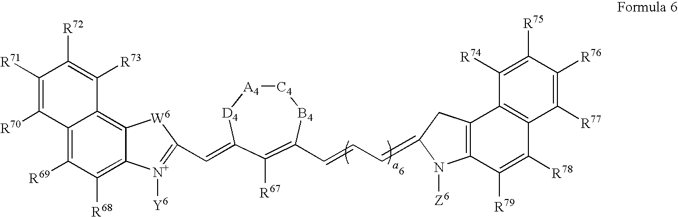

W⁶ and X⁶ may be the same or different and are selected from the group consisting of —CR¹R², —O—, —NR³, —S—, and —Se; Y⁶ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H, —(CH₂)ₐ—CONH₂, —CH₂(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂—(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂—(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; Z⁶ is selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, —CH₂(CH₂OCH₂)_{b}—CH₂—OH, —(CH₂)ₐ—CO₂H—(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R³)—(CH₂)_{b}—CONH₂, (CH₂)ₐ—N(R³)—(CH₂)_{c}—NHCOH, —(CH₂)ₐ—N(R³)—CH₂—(CH₂OCH₂)_{b}—CH₂—CONH₂, —(CH₂)ₐ—N(R³)—CH₂—(CH₂OCH₂)_{b}—CH₂—NHCOH, —CH₂—(CH₂OCH₂)_{b}—CH₂—N(R³)—(CH₂)ₐ—CONH₂, —CH₂(CH₂OCH₂)_{b}—CH₂—N(R³)—(CH₂)ₐ—NHCOH, —CH₂—(CH₂OCH₂)_{b}—CH₂—N(R³)—CH₂—(CH₂OCH₂)_{d}—CONH₂, —CH₂(CH₂OCH₂)_{b}—CH₂—N(R³)—CH₂—(CH₂OCH₂)_{d}—NHCOH, —(CH₂)ₐ—NR³R⁴, and —CH₂(CH₂OCH₂)_{b}—CH₂NR³R⁴; A₄ is a single or a double bond; B₄, C₄, and D₄ may be the same or different and are selected from the group consisting of —O—, —S—, —Se—, —P—, —CR¹R², —CR¹, alkyl, NR³, and —C═O; A₄, B₄, C₄, and D₄ may together form a 6- to 12-membered carbocyclic ring or a 6- to 12-membered heterocyclic ring optionally containing one or more oxygen, nitrogen, or sulfur atom; a₆ is independently from 0 to 5; R¹ to R⁴, and R⁶⁷ to R⁷⁹ are independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, glucose derivatives of R groups, cyano, nitro, halogen, saccharide, —CH₂(CH₂OCH₂)_{b}—CH₂OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—OH or —CH₂—(CH₂OCH₂)_{b}—CO₂H; a and c are independently from 1 to 20; and b and d are independently from 1 to 100.

Even yet another exemplary family of optical agents that may be utilized in processes of the invention include optical agents corresponding to formula 7 below, wherein

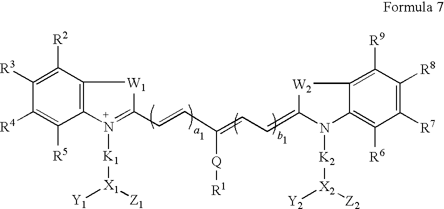

W₁ and W₂ may be the same or different and are selected from the group consisting of —CR¹⁰R¹¹, —O—, —NR¹², —S—, and —Se; Y₁, Y₂, Z₁, and Z₂ are independently selected from the group consisting of hydrogen, —CONH₂, —NHCOH, —(CH₂)ₐ—CONH₂, —CH₂(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R¹²)—(CH₂)_{b}—CONH₂, —(CH₂)ₐ—N(R¹²)—(CH₂)_{c}—NHCOH, —(CH₂)ₐ—N(R¹²)—CH₂—(CH₂OCH₂)_{b}—CH₂—CONH₂, —(CH₂)ₐ—N(R¹²)—CH₂—(CH₂OCH₂)_{b}—CH₂—NHCOH, —CH₂—(CH₂OCH₂)_{b}—CH₂—N(R¹²)—(CH₂)ₐ—CONH₂, —CH₂—(CH₂OCH₂)_{b}—CH₂N(R¹²)—(CH₂)ₐ—NHCOH, —CH₂—(CH₂OCH₂)_{b}—CH₂—N(R¹²)—CH₂—(CH₂OCH₂)_{d}—CONH₂, —CH₂—(CH₂OCH₂)_{b}—CH₂—N(R¹²)—CH₂—(CH₂OCH₂)_{d}—NHCOH, —CONH₂, —NHCOH, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—N(R¹²)—(CH₂)_{b}—CONH₂, —(CH₂)ₐ—N(R¹²)—(CH₂)_{c}—NHCOH, —(CH₂)ₐ—N(R¹²)—CH₂—(CH₂OCH₂)_{b}—CH₂CONH₂, —(CH₂)ₐ—N(R¹²)—CH₂—(CH₂OCH₂)_{b}—CH₂—NHCOH, —CH₂(CH₂OCH₂)_{b}—CH₂—N(R¹²)—(CH₂)ₐ—CONH₂, —CH₂—(CH₂OCH₂)_{b}—CH₂—N(R¹²)—(CH₂)ₐ—NHCOH, —CH₂—(CH₂OCH₂)_{b}—CH₂—N(R¹²)—CH₂—(CH₂OCH₂)_{d}—CONH₂, —CH₂(CH₂OCH₂)_{b}—CH₂—N(R¹²)—CH₂—(CH₂OCH₂)_{d}—NHCOH, —(CH₂)ₐ—NR¹²R¹³, and —CH₂(CH₂OCH₂)_{b}—CH₂NR¹²R¹³; K₁ and K₂ are independently selected from the group consisting of C₁-C₃₀ alkyl, C₅-C₃₀ aryl, C₁-C₃₀ alkoxyl, C₁-C₃₀ polyalkoxyalkyl, C₁-C₃₀ polyhydroxyalkyl, C₅-C₃₀ polyhydroxyaryl, C₁-C₃₀ aminoalkyl, saccharides, —CH₂(CH₂OCH₂)_{b}—CH₂—, —(CH₂)ₐ—CO—, —(CH₂)ₐ—CONH—, —CH₂—(CH₂OCH₂)_{b}—CH₂—CONH—, —(CH₂)ₐ—NHCO—, —CH₂—(CH₂OCH₂)_{b}—CH₂—NHCO—, —(CH₂)ₐ—O—, and —CH₂—(CH₂OCH₂)_{b}—CO—; X₁ and X₂ are single bonds, or are independently selected from the group consisting of nitrogen, saccharides, —CR¹⁴—, —CR¹⁴R¹⁵, —NR¹⁵R¹⁷; C₅-C₃₀ aryl; Q is a single bond or is selected from the group consisting of —O—, —S—, —Se—, and —NR¹⁸; a₁ and b₁ independently vary from 0 to 5; R¹ to R¹³, and R¹⁸ are independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, cyano, nitro, halogens, saccharides, —CH₂(CH₂OCH₂)_{b}—CH₂—OH, —(CH₂)ₐ—CO₂H, —(CH₂)ₐ—CONH₂, —CH₂—(CH₂OCH₂)_{b}—CH₂—CONH₂, —(CH₂)ₐ—NHCOH, —CH₂—(CH₂OCH₂)_{b}—CH₂—NHCOH, —(CH₂)ₐ—OH and —CH₂—(CH₂OCH₂)_{b}—CO₂H; R¹⁴ to R¹⁷ are independently selected from the group consisting of hydrogen, C₁-C₁₀ alkyl, C₅-C₂₀ aryl, C₁-C₁₀ alkoxyl, C₁-C₁₀ polyalkoxyalkyl, C₁-C₂₀ polyhydroxyalkyl, C₅-C₂₀ polyhydroxyaryl, C₁-C₁₀ aminoalkyl, saccharides, —CH₂(CH₂OCH₂)_{b}—CH₂—, —(CH₂)ₐ—CO—, —(CH₂)ₐ—CONH—, —CH₂—(CH₂OCH₂)_{b}—CH₂—CONH—, —(CH₂)ₐ—NHCO—, —CH₂—(CH₂OCH₂)_{b}—CH₂—NHCO—, —(CH₂)ₐ—O—, and —CH₂—(CH₂OCH₂)_{b}—CO—; a and c independently vary from 1 to 20; b and d independently vary from 1 to 100.

Another exemplary family of optical agents that may be utilized in processes of the invention include optical agents corresponding to formula 8 below, wherein

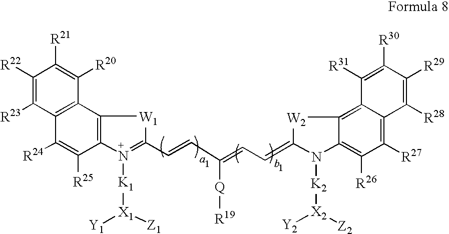

W₁, W₂, Y₁, Y₂, Z₁, Z₂, K₁, K₂, Q, X₁, X₂, a₁, and b₁ are defined in the same manner as in Formula 1; and R¹⁹ to R³¹ are defined in the same manner as R¹ to R⁹ in Formula 7.

Still another exemplary family of optical agents that may be utilized in processes of the invention include optical agents corresponding to formula 9 below, wherein

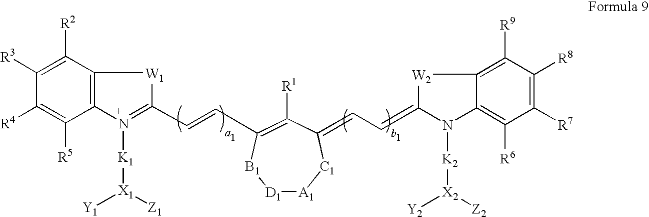

A₁ is a single or a double bond; B₁, C₁, and D₁ are independently selected from the group consisting of —O—, —S—, —Se—, —P—, —CR¹⁰R¹¹, —CR¹¹, alkyl, NR¹², and —C═O; A₁, B₁, C₁, and D₁ may together form a 6- to 12-membered carbocyclic ring or a 6- to 12-membered heterocyclic ring optionally containing one or more oxygen, nitrogen, or sulfur atoms; and W₁, W₂, Y₁, Y₂, Z₁, Z₂, K₁, K₂, X₁, X₂, a₁, b₁, and R¹ to R¹² are defined in the same manner as in Formula 7.

Yet another exemplary family of optical agents that may be utilized in processes of the invention include optical agents corresponding to formula 10 below, wherein

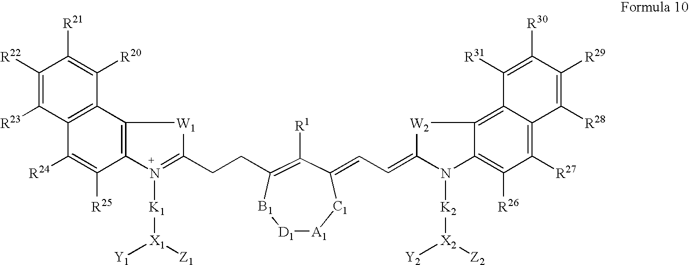

A₁, B₁, C₁, and D₁ are defined in the same manner as in Formula 9; W₁, W₂, Y₁, Y₂, Z₁, Z₂, K₁, K₂, X₁, X₂, a₁, and b₁ are defined in the same manner as in Formula 7; and R¹⁹ to R³¹ are defined in the same manner as R¹ to R⁹ in Formula 7.

Other optical agents that may be used as optical agents in the processes of the present invention include, but are not limited to, for example, fluorescein and indocyanine (NIRD)-polyaspartic acid 6000 conjugates. Methods for the preparation of these compounds are described in U.S. Pat. No. 6,228,344. Examples of other optical agents that may be utilized in the processes of the invention include Bis(hexanoic acid)indocyanine green-polyaspartic acid, Bis(hexanoic acid)indocyanine green-polyglutamic acid, Bis(hexanoic acid)indocyanine green-polyacrylic acid, Bis(hexanoic acid)indocyanine green-polynucleotides, Bis(hexanoic acid)indocyanine green-polynitrophenylalanine, Bis(hexanoic acid)indocyanine green-polydinitrophenylalanine, Bis(hexanoic acid)indocyanine green-polytrinitrophenylalanine, Bis(hexanoic acid)indocyanine green-polysulfonylphenylalanine, Bis(hexanoic acid)indocyanine green-polydisulfonylphenylalanine, Bis(hexanoic acid)indocyanine green-polytrisulfonylphenylalanine, Bis(hexanoic acid)indocyanine green-polysuccinate, Bis(hexanoic acid)indocyanine green-polymalonate, Bis(hexanoic acid)indocyanine green-polyglutarate, Bis(hexanoic acid)indocyanine green-polyglycolate, Bis(propanoic acid)indocyanine green-polyaspartic acid, Bis(propanoic acid)indocyanine green-polyglutamic acid, Bis(propanoic acid)indocyanine green-polyacrylic acid, Bis(propanoic acid)indocyanine green-polynucleotides, Bis(propanoic acid)indocyanine green-polynitrophenylalanine, Bis(propanoic acid)indocyanine green-polydinitrophenylalanine, Bis(propanoic acid)indocyanine green-polytrinitrophenylalanine, Bis(propanoic acid)indocyanine green-polysulfonylphenylalanine, Bis(propanoic acid)indocyanine green-polydisulfonylphenylalanine, Bis(propanoic acid)indocyaninegreen-polytrisulfonylphenylalanine, Bis(propanoic acid)indocyanine green-polysuccinate, Bis(propanoic acid)indocyanine green-polymalonate, Bis(propanoic acid)indocyanine green-polyglutarate, and Bis(propanoic acid)indocyanine green-polyglycolate

### III. Routes of Administration

Effective amounts of one or more optical agents can be administered to a surgical patient by any of number of various processes known in the art. An optical agent may be administered parenterally or enterally. In some embodiments, one or more optical agents are administered systemically for delivery to the biliary tract of a patient. For example, optical agents can be administered to a patient intravenously, intraarterially, orally, via a gastric or intestinal (e.g., duodenal or jejunal) feeding tube, by intramuscular injection, by subcutaneous injection or infusion, by intraperitoneal injection or infusion, intrathecally, sublingually, rectally, vaginally, nasally, by inhalation, by transdermal absorption through the skin, and/or by intraosseous infusion. Preferably, optical agents are administered orally or intravenously in processes of the invention.

Intravenous administration may be used to deliver a single dose or bolus of one or more optical agents. Alternatively, intravenous administration of the optical agent(s) can be intermittent or continuous (e.g., infusion).

In some embodiments, optical agents are administered locally to a patient's biliary tract or a tissue thereof via an appropriate delivery device. For example, one or more optical agents may be injected directly into a tissue of the biliary tract.

The delay between administration of the optical agent(s) and appearance of the optical agent(s) in a patient's biliary tract may vary depending on the specific optical agent(s) involved, the route of administration, the route by which the agent is primarily excreted (i.e., hepatobiliary or renal), and the like.

Administration of more than one optical agent to a surgical patient can be accomplished by administering a formulation (e.g., a sterile solution for intravenous or intraperitoneal administration) containing all of the optical agents to be administered. Alternatively, each optical agent may be administered in a separate formulation. When more than one optical agent is administered to a patient, administration of each agent need not be via the same route (e.g., one agent could be administered by orally while another is administered intravenously). Administration of multiple optical agents may be, but need not be, simultaneous.

The hepatobiliary cleared optical agents can be co-administered with other biocompatible compounds.

### IV. Formulations

Hepatobiliary cleared optical agents utilized in processes of the invention can be formulated into compositions for enteral or parenteral administration to a patient. In general, such compositions may contain an effective amount of one or more optical agents, along with pharmaceutical carriers and excipients appropriate for the desired route of administration. The composition may thus contain a single optical agent or may contain a plurality of optical agents for co-administration to a patient.

In some embodiments, the compositions that contain the optical agent(s) is/are formulated as sterile aqueous solutions or suspensions for parenteral administration. Such parenteral solutions or suspensions may be injected directly or mixed with a large volume parenteral composition for systemic administration. Exemplary routes for administration of such solutions include intravenous administration, intraperitoneal injection, and infusion.

Sterile aqueous solutions or suspensions for parenteral administration that contain one or more hepatobiliary cleared optical agents may optionally contain one or more of the following components: pharmaceutically acceptable buffers, electrolytes (e.g., sodium chloride), diluents, solvents, antimicrobial agents, chelating agents (e.g., ethylenediaminetetraacetic acid (EDTA)), preservatives, surfactants, and/or any other appropriate biocompatible compound(s).

In some embodiments, optical agents may be formulated for enteral administration, for example, as sterile aqueous solutions or suspensions or as solids. Optical agents formulated in a sterile aqueous solution or suspension for enteral administration may be administered, for instance, orally or via a feeding tube. Such solutions or suspensions may optionally contain one or more of the following components in addition to the optical agent(s): pharmaceutically acceptable buffers, electrolytes (e.g., sodium chloride), diluents, solvents, antimicrobial agents, chelating agents (e.g., ethylenediaminetetraacetic acid (EDTA)), preservatives, surfactants, thixotropic agents, and/or any other appropriate biocompatible compound. Aqueous solutions for oral administration may optionally contain flavoring agents and/or other ingredients for enhancing their organoleptic qualities.

Optical agents utilized in processes of the invention may be formulated as solids for oral administration. For example, optical agents may be enclosed in capsules or compressed into tablets. Solid formulations for oral administration containing one or more hepatobiliary cleared optical agents may optionally contain one or more of the following components: binders (e.g., a starch, sugar, cellulose, or sugar alcohol), fillers (e.g., a plant cellulose, dibasic calcium phosphate, soybean oil, or safflower oil), disintegrants, lubricants (e.g., stearic acid or magnesium stearate), coatings (e.g., cellulose or a synthetic polymer), sweeteners or other flavoring agents, preservatives, and/or any other appropriate biocompatible compound(s).

In some embodiments, optical agents may be formulated as solids to be reconstituted into a sterile aqueous solution or suspension prior to administration.

Optical agents to be utilized in some processes of the invention may be formulated in liposomes, micelles, microemulsions, microspheres, microcapsules, or any of a number of nano-sized entities (e.g., nanoparticles, nanospheres, nanocapsules, nanorods, nanoeggs, etc.). Preparation and loading may be accomplished by processes known in the art.

### V. Dosing

The amount of optical agent administered for a surgical procedure will typically depend upon the identity of the optical agent, the route of administration, the means employed for detection, the tissue(s) to be delimited, the degree of fluorescence desired, and the surgical method employed. By way of example, dosages of some embodiments may range from about 0.05 μm/kg body weight to about 20 μm/kg of body weight.

### VI. Kit

For convenience, optical agents for use in processes of the invention may be provided to a user in the form of a kit containing some or all of the necessary components. The kit may include one or more of the following components: (i) one or more optical agents, (ii) means for administration (e.g., syringe), and (iii) instructions for using the optical agent(s) to optically detect one or more tissues of the biliary tract of a surgical patient. The kit may optionally contain one or more biocompatible solvents, buffers, excipients, salts, preservatives, and the like.

In some embodiments, an optical agent is provided in the kit as a sterile aqueous solution or suspension that can be administered, for example, intravenously, by intraperitoneal injection or infusion, or in any other appropriate manner. In some embodiments, the optical agent may be provided in the kit as a sterile aqueous solution or suspension for oral administration. In some embodiments, the optical agent may be provided in the kit as a solid composition (e.g., a tablet or capsule) that can be administered orally. In some embodiments, the optical agent may be provided in the kit as a solid formulation for reconstitution into a sterile aqueous solution or suspension prior to administration to a surgical patient.

The instructions of the kit may include, for example, information about the optical agent(s) (e.g., dosage information, optimal absorption or excitation wavelengths, optimal detection wavelengths, hepatobiliary clearance kinetics, optimal timing of administration with relation to the surgical procedure, and the like), information regarding any other compounds included in the formulation (e.g., buffers, diluents, preservatives, etc., as described above), instructions for reconstituting a solid biocompatible composition included in the kit, instructions for administering the biocompatible composition of the kit to a surgical patient, instructions for detecting an optical agent following administration to a surgical patient, instructions for optimizing detection of the optical agent(s), and/or instructions for determining the extent of hepatobiliary excretion of the optical agent(s).

### Example 1

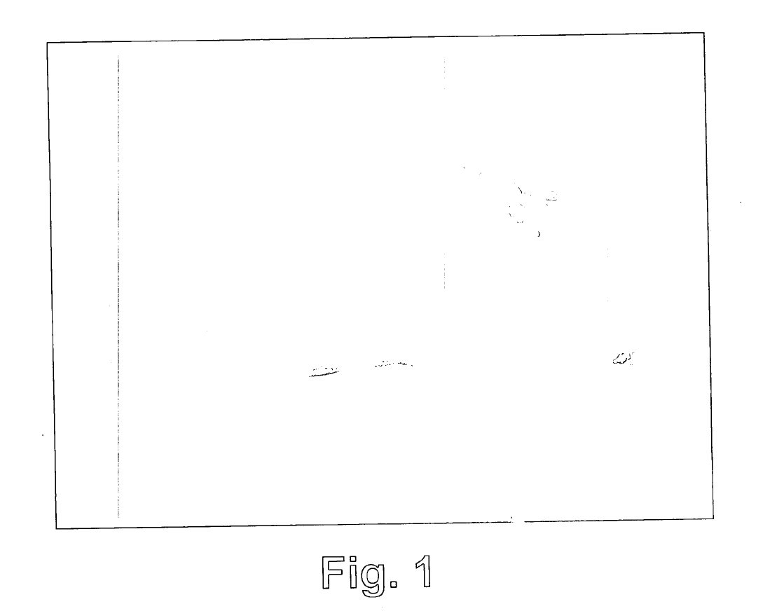

FIG. 1 is a digital camera image of fluorescence from fluorescein traveling through the cystic duct of an anesthetized pig. The image was taken during open surgery, approximately 80 minutes post-IV administration of 6 mL of a 20 mg/mL concentration of fluorescein in PBS. The pig weighed about 82 pounds.

### Example 2

ICG in powdered form was obtained from Sigma (St. Louis, Mo.). Solutions were made by diluting the appropriate amount of ICG in 10 mL of distilled water. Concentrations less than 1 mM were made by serial dilution of a 1 mM stock solution.

The animals were anesthetized with rat cocktail (xylazine; ketamine; acepromazine 1.5: 1.5: 0.5) at 0.8 mL/kg via intraperitoneal injection. A 21 gauge butterfly infusion set equipped with a stopcock and two syringes containing heparinized saline was placed into the lateral tail vein of the rat. Patency of the vein was checked prior to administration of the agent via the butterfly apparatus.

A simple noninvasive in vivo continuous wave fluorescence imaging apparatus was employed as described below. Light from a LaserMax Inc. laser diode of nominal wavelength 780 nm and nominal power of 40 mW was launched into a fiber optic bundle. A defocusing lens in position after the bundle expanded the beam such that most of the rat was illuminated. The laser power at the output of the bundle was approximately one half of the input power. The detector was a Princeton Instruments model RTE/charge coupled device (CCD)-1317-K/2 CCD camera with a Rodenstock 10 mm F2 lens (stock No. 542.032.002.20) attached. An 830 nm interference lens (CVI Laser Corp. part No. F10-830-4-2) was mounted in front of the CCD input lens such that only emitted fluorescent light from the contrast agent was imaged. Images were acquired and processed using WinView software from Princeton Instruments. An image of the animal was taken pre-administration of ICG. Subsequently, images were typically taken at 0.5, 1, 2, 5, 10, 20, 30, 45, 60, and 90 min post-administration of the agent, all performed with the rat in a stationary position. Data analysis consisted of subtracting (pixel by pixel) the pre-administration image from the post-administration images, and displaying the false color results. An approximate 24 h time point was also imaged; however, the subtraction of the original background was not performed since the animal had been removed from the sample area and returned at this later time.

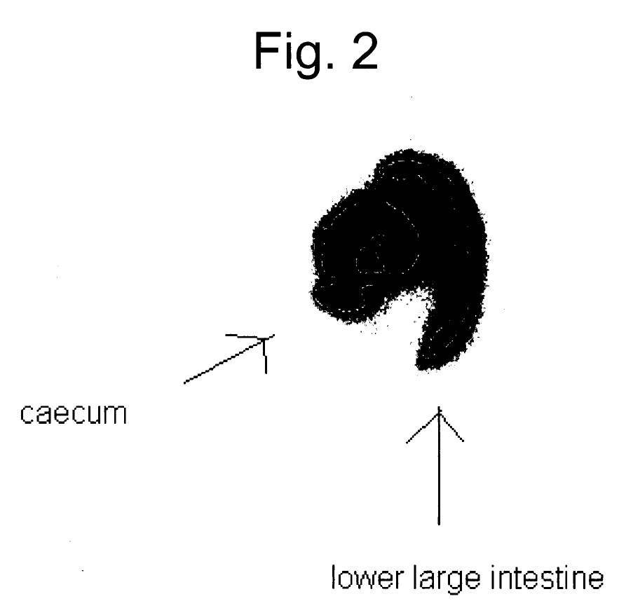

The above imaging system may also used to view and quantify ex-vivo organs and tissues. In FIG. 2, the caecum and lower large intestine of a rat is displayed, post-IV administration of an aqueous ICG solution. It is well known that ICG is removed from the bloodstream by the liver, goes through the bile, and is excreted in the feces. FIG. 2 shows ex-vivo tissues of a rat with feces that fluoresce due to the ICG within. Blue is indicative of high fluorescence, and red is indicative of low fluorescence.

### Example 3

ICG in powdered form was obtained from Sigma (St. Louis, Mo.). Solutions were made by diluting the appropriate amount of ICG in 10 mL of distilled water. Concentrations less than 1 mM were made by serial dilution of a 1 mM stock solution. For ICG fluorescence detection, a nominal 780 nm collimated solid state laser source was employed (LaserMax Inc. model No. LAS-300-780-5). The laser source was directed into the end of a 3.2 mm diameter glass fiber optic bundle (Oriel No. 77526). The other end of this laser delivery bundle was placed approximately 1 cm from the rat ear at an approximate 45° angle. A second similar fiber optic bundle for use as the fluorescence detection conduit was placed approximately 1 cm from the ear at an approximate 30° angle. The exit end of the detection fiber bundle was positioned at the focal length of a 20 mm focal length lens. The output light was thus directed toward the detector after exiting the bundle and passing through the lens. A narrow band interference filter was the next element in the optics train (CVI Laser Corporation), allowing light of the appropriate wavelength to pass on to the detector. An 830 nm filter [10 nm full width at half maximum (FWHM) bandwidth] was used. The detector was a small silicon photodiode (UDT model PIN-10DP) connected to a transimpedance amplifier (Graseby Optronics model TRAMP®). A digital voltmeter monitored the output signal. A subsequent voltage amplifier (Tektronix AM-502) boosted the signal if needed. The amplifier output was connected to a National Instruments BNC-2080 breakout board, which was interfaced to a National Instruments DAQCard-700 data acquisition board (A/D). LabVIEW® data acquisition software collects the experimental raw data.

Female Fischer 344 rats weighing 173-195 g were used. These animals were first anesthetized with urethane (1.35 g/kg) administered via intraperitoneal injection. The anesthesia dose was administered so as to minimize the variability of the anesthetic plane achieved by the individual rats. After the animals had achieved the desired plane of anesthesia, a 21 gauge butterfly with 12 inch tubing was placed in the lateral tail vein of each animal and flushed with heparinized saline. The animals were placed onto a heating pad and kept warm throughout the entire study. The lobe of the left ear was fixed to a glass microscope slide. The dye was subsequently administered via the indwelling catheter, and the clearance of the agent monitored. Clearance curves were obtained from an n=3 or 6 population.

To verify that the fluorescence decay curves were related to liver function, and to assess this methodology's feasibility to determine impaired liver function, the following experiment was done. A partial hepatectomy was surgically performed on three rats as described in H. B. Waynforth and P. A. Flecknell, Experimental and Surgical Technique in the Rat, p. 241, Academic, London (1992). Once the surgery was complete, each rat was allowed to equilibrate for 10 minutes and then injected with 500 mL of 1.007 mM ICG solution. A measurement of the time dependence of fluorescence at the ear pre- and post-bolus injection of the ICG solution was measured next. Two of these data sets are shown in FIG. 5, along with a measurement from a normal liver functioning rat for comparison.

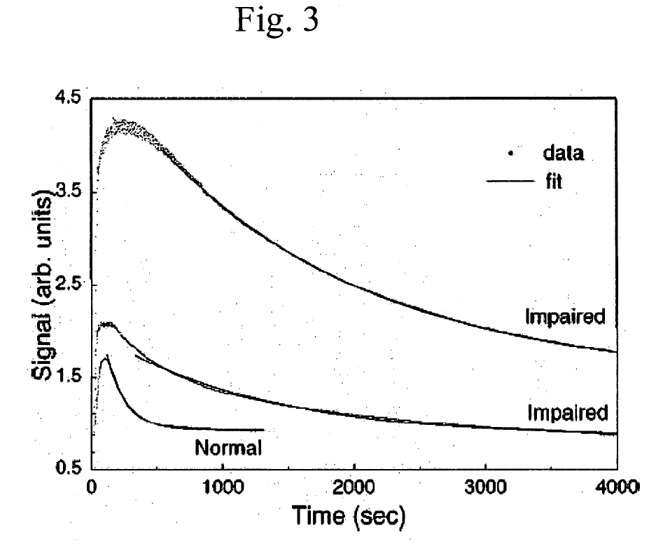

A measurement of the time dependence of fluorescence at the ear pre- and post-bolus injection of the ICG solution can be described in terms of three stages. Stage 1 consisted of approximately the first 30 seconds of data, which was gathered at the time of the pre-bolus injection. Stage 2 occurred several seconds post-injection with the signal rapidly rising to a maximum as the dye was reaching the ear and equilibrating in the blood pool. In the third stage, the fluorescence signal decayed with time as the liver filtered the ICG out of the blood stream. Visually, the decay rates were similar for all three data sets, and well within biological variability (see FIG. 3). As can be seen from the same figure, approximately 90% of the initial signal was lost after about 15 minutes. The apparent rapid distribution (equilibration) of ICG into the bloodstream (steep rise of Stage 2 in any of the data sets) and the apparent exponential decay of ICG from the bloodstream (Stage 3 data) suggest that the data may follow an open one compartment pharmacokinetic model. The characteristics of such a model are no absorption, entire drug dose in systemic circulation, rapid distribution of drug between bloodstream and tissue, instantly attained equilibrium (steady state), and that the drug concentration decrease is dependent on excretion. The fluorescent signal (arising from the dye concentration in the blood) as a function of time was fit to a single exponential decay appropriate to an open one compartment pharmacokinetic model. The equation employed to fit the Stage 3 data was S=Ae^{-t/τ}+B (Eq. 1), where S is the fluorescent light intensity signal measured, and t is the time point of the measurement. The quantity of interest τ, which is the decay time, and constants A and B are deduced from the fitting procedure. The nonlinear regression analysis package within SigmaPlot® (Jandel Scientific Software, Rafael, Calif.) was employed to fit data to Eq. 1. As can be seen from FIG. 3, the capability of the liver to remove ICG from the blood pool was reduced in partially hepatectomized rats. The fluorescence decay rate was much slower in the rats with partially hepatectomized livers than that of normal liver functioning rats. The decay time for the impaired liver function was almost an order of magnitude longer than the normal liver function decay time. Upon sacrifice, the livers were extracted and weighed. The amount of ligated liver (nonfunctional) ranged from 33%-38% of the total liver weight. Thus, an order of magnitude change in the decay time resulted from a reduction of the functioning liver mass by approximately one-third. The capability to discriminate an even smaller reduction in functioning liver mass by this technique may be reasonably expected.

When introducing elements of the present invention or the exemplary embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above processes and kits without departing from the scope of the invention, it is intended that all matter contained in the above description shall be interpreted as illustrative and not in a limiting sense.

1-12. (canceled)

13. A process for using an optical agent in a surgical procedure, the process comprising:
delivering a hepatobiliary cleared optical agent to a patient to cause the optical agent to appear in the patient's biliary tract;
irradiating a first tissue of the patients biliary tract with non-ionizing radiation to detect the optical agent, wherein the first tissue is selected from the group consisting of common bile duct, hepatic duct, cystic duct, and combinations thereof; and
detecting the agent in the irradiated first tissue to demarcate the position of the first tissue.

14. The process of claim 13, wherein the delivering is performed by intravenous injection.

15. The process of claim 13, wherein the detecting is performed using at least one of unaided eye, camera, charged coupled device, photomultiplier tube, avalanche diode and photodiode.

16. The process of claim 13, wherein the non-ionizing radiation is selected from the group consisting of visible radiation, ultraviolet radiation, infrared radiation, and combinations thereof.

17. The process of claim 13, wherein the surgical procedure is selected from the group consisting of cholecystectomy, liver transplantation, liver resection, total or partial hysterectomy, hernia repair surgery, colectomy, appendectomy, splenectomy, distal or total pancreatectomy, the Whipple procedure, removal of inflammatory or malignant tumors in the abdominal regions, and abdominal lymphadenectomy.

18. A kit comprising:
a hepatobiliary cleared optical agent; and
instructions for using the agent in the process of claim 13.

19. The kit of claim 18, further comprising:
at least one substance selected from the group consisting of biocompatible solvents, buffers, excipients, salts, preservatives, and flavoring agents.

20. The kit of claim 18, wherein the agent is provided in the kit as a sterile aqueous solution or suspension.

21. The kit of claim 18, wherein the agent is provided in the kit as a solid composition or solid formulation.

22. A process for using an optical agent in a surgical procedure, the process comprising:
delivering a hepatobiliary cleared optical agent to a patient to cause the optical agent to appear in a first tissue of the patient's biliary tract, wherein the first tissue is selected from the group consisting of common bile duct, hepatic duct, cystic duct, and combinations thereof;
irradiating the first tissue with non-ionizing radiation;
at least one of during and subsequent to the delivering, detecting the optical agent based, at least in part, on the irradiating; and
determining if the agent is retained within the first tissue based, at least in part, on the detecting.

23. The process of claim 22, wherein the delivering is performed by intravenous injection.

24. The process of claim 22, wherein the detecting is performed using at least one of unaided eye, camera, charged coupled device, photomultiplier tube, avalanche diode and photodiode.

25. The process of claim 22, wherein the non-ionizing radiation is selected from the group consisting of visible radiation, ultraviolet radiation, infrared radiation, and combinations thereof.

26. The process of claim 22, wherein the surgical procedure is selected from the group consisting of cholecystectomy, liver transplantation, liver resection, total or partial hysterectomy, hernia repair surgery, colectomy, appendectomy, splenectomy, distal or total pancreatectomy, the Whipple procedure, removal of inflammatory or malignant tumors in the abdominal regions, and abdominal lymphadenectomy.

27. A kit comprising:
a hepatobiliary cleared optical agent; and
instructions for using the agent in the process of claim 22.

28. The kit of claim 27, further comprising:
at least one substance selected from the group consisting of biocompatible solvents, buffers, excipients, salts, preservatives, and flavoring agents.

29. The kit of claim 28, wherein the agent is provided in the kit as a sterile aqueous solution or suspension.

30. The kit of claim 28, wherein the agent is provided in the kit as a solid composition or solid formulation.

31. A process for using an optical agent in a surgical procedure, the process comprising:
irradiating a surgical field of a patient with non-ionizing radiation to detect a hepatobiliary cleared optical agent, which is located in a first tissue of the biliary tract in the surgical field of the patient during the irradiating, wherein the first tissue is selected from the group consisting of common bile duct, hepatic duct, cystic duct, and combinations thereof; and
surgically manipulating a second tissue of the patient based, at least in part, on optical detection of the agent within the first tissue.

32. The process of claim 31, wherein the surgically manipulating comprises surgically manipulating the second tissue of the patient based, at least in part, on optical detection of the agent within the first tissue using at least one of unaided eye, camera, charged coupled device, photomultiplier tube, avalanche diode, and photodiode.

33. The process of claim 31, wherein the non-ionizing radiation is selected from the group consisting of visible radiation, ultraviolet radiation, infrared radiation, and combinations thereof.

34. The process of claim 31, wherein the surgical procedure is selected from the group consisting of cholecystectomy, liver transplantation, liver resection, total or partial hysterectomy, hernia repair surgery, colectomy, appendectomy, splenectomy, distal or total pancreatectomy, the Whipple procedure, removal of inflammatory or malignant tumors in the abdominal regions, and abdominal lymphadenectomy.

35. A kit comprising:
a hepatobiliary cleared optical agent; and
instructions for using the agent in the process of claim 31.

36. The kit of claim 35, further comprising:
at least one substance selected from the group consisting of biocompatible solvents, buffers, excipients, salts, preservatives, and flavoring agents.

37. The kit of claim 35, wherein the agent is provided in the kit as a sterile aqueous solution or suspension.

38. The kit of claim 35, wherein the agent is provided in the kit as a solid composition or solid formulation.
